(19) United States
(12) Patent Application Publication
(10) Pub. No.: US 2008/0004503 A1
(43) Pub. Date: Jan. 3, 2008

(54) Data recorder and method for recording a data signal received from an in-vivo sensing device

(76) Inventors: Micha Nisani, Ramot Itzhak (IL); Ido Bettesh, Haifa (IL); Pesach Pascal, Nesher (IL); Uri Kogan, Nesher (IL); Ofra Zinaty, Haifa (IL); Tal Davidson, Yoqneam Illit (IL); Eli Horn, Kiryat Motzkin (IL)

Correspondence Address: PEARL COHEN ZEDEK LATZER, LLP, 1500 BROADWAY 12TH FLOOR, NEW YORK, NY, 10036, US

(21) Appl. No.: 11/476,903

(22) Filed: Jun. 29, 2006

(51) Int. Cl. A61B 5/00

(52) U.S. Cl. 600/300

(57) ABSTRACT

A data recorder adapted for processing sensed data received from an in-vivo sensing device. The sensed data may be image data of images captured by an imager of the in-vivo sensing device and the processing may be image processing.

### FIELD OF THE INVENTION

The present invention relates in general to a data recorder for receiving data transmitted by an in-vivo sensing device. More specifically, the present invention relates to processing of the received data by the data recorder.

### BACKGROUND OF THE INVENTION

In-vivo sensing devices for diagnosis of the gastrointestinal (GI) tract or other body lumens of a patient such as, for example, ingestible sensing capsules, may wirelessly transmit sensed data, such as imaging data, to an external data recorder. The data recorder may be affixed to the patient by a strap or a belt so that the patient may freely perform normal actions during an observation period that may begin after swallowing of the in-vivo sensing device and end upon its excretion. The data recorder may have radio communication capability and it may have connected to it one or more antennas for receiving the sensed data transmitted by the in-vivo sensing device and the data recorder may have a memory for storing the received sensed data. After the observation period, the patient may deliver the data recorder to an operator, for example, a health professional who may download the stored sensed data for processing and for performing analysis of the GI tract for diagnosis purposes. The sensed data may include image data of images of the GI tract captured by an imager in the in-vivo sensing device as it passes through the GI tract.

The sensed data may be downloaded from the data recorder to a workstation, or the like, in order to analyze the images of the GI tract for diagnosis purposes. After the sensed data is downloaded to the workstation the image data has to undergo various forms of image processing in the workstation before the images can be diagnosed. However, the image processing is time consuming thereby delaying the start of diagnosis from the time the sensed data is downloaded.

### SUMMARY OF THE INVENTION

According to embodiments of the present invention some of the processing of the downloaded sensed data that may normally be performed in the workstation may be performed in the data recorder. The processing may be image processing or other signal processing, or both. If desired, the processing that may be performed in the data recorder may be processing that is not computationally demanding and time consuming. By performing some of the processing in the data recorder, the time spent on image processing in the work station is reduced, thereby enabling image diagnosis in the workstation to commence in a shorter time after downloading the image data from the data recorder, in comparison with the situation in which all the image processing is performed in the workstation.

According to one embodiment of the present invention, there is provided a data recorder for recording a data signal received from an in-vivo sensing device, the data recorder comprising a modem, a telemetry extraction unit, a header extraction unit, and a storage unit, the data signal comprising image data representing images captured by an imager of the in-vivo sensing device.

According to some embodiments, the data recorder comprises a deinterleaving unit.

According to some embodiments, the data recorder comprises an error correction unit.

If desired, the error correction unit comprises a convolution decoder.

Further if desired, the error correction unit comprises a block code decoder.

According to some embodiments, the data recorder comprises a decompression unit for decompressing compressed data.

According to some embodiments, the data recorder comprises a mosaic to RGB un it for converting a mosaic representation of image data to an RGB representation.

If desired, the data recorder comprises a color bar generator for generating a color bar for aiding in identifying physical regions of the images captured by the imager.

According to some embodiments, the data recorder comprises a localization calculation unit for determining the location of the in-vivo sensing device.

According to some embodiments, the data recorder comprises an image combining unit to combine image frames into a single image.

According to another embodiment of the present invention, there is provided a method for recording a data signal received from an in-vivo sensing device comprising:

demodulating the data signal;

extracting telemetry and header information from the demodulated signal; and

storing the demodulated signal and the telemetry and header information.

According to some embodiments, the method comprises the further step of deinterleaving the demodulated signal.

According to some embodiments, the method comprises the further step of error correction of the deinterleaved signal.

If desired, the step of error correction comprises convolution decoding.

Further if desired, the step of error correction comprises block code correction.

According to some embodiments, the method comprises the further step of decompressing the error corrected signal.

According to some embodiments, the method comprises the step of generating RGB images from mosaic images of the decompressed signal.

If desired, the method comprises the further step of generating a color bar for the decompressed data signal.

According to some embodiments, the method comprises the further step of performing a localization calculation on the decompressed data signal to determine the location of the in-vivo sensing device.

According to some embodiments, the method comprises the further step of combining at least some decompressed individual images into a combined single image.

### BRIEF DESCRIPTION OF THE DRAWINGS

The present invention will be understood and appreciated more fully from the following detailed description taken in conjunction with the drawings in which:

FIG. 1 is a simplified conceptual illustration of an in-vivo imaging system according embodiments of the present invention; and

FIG. 2 is an illustrative block diagram showing components of a data recorder in accordance with some embodiments of the present invention.

It will be appreciated that for simplicity and clarity of illustration, elements shown in the figures have not necessarily been drawn accurately or to scale. For example, the dimensions of some of the elements may be exaggerated relative to other elements for clarity, or several physical components may be included in one functional block or element. Further, where considered appropriate, reference numerals may be repeated among the figures to indicate corresponding or analogous elements.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well-known features may be omitted or simplified in order not to obscure the present invention.

The device, system and method of the present invention may be used with an imaging system or device such as that described in U.S. Pat. No. 5,604,531 entitled “In Vivo Video Camera System,” which is incorporated herein by reference. A further example of an imaging system and device with which the system and method of the present invention may be used is described in U.S. Pat. No. 7,009,634 entitled “Device for In Vivo Imaging,” which is incorporated herein by reference. For example, a swallowable imaging capsule such as that described in U.S. Pat. No. 7,009,634, may be used in the present invention.

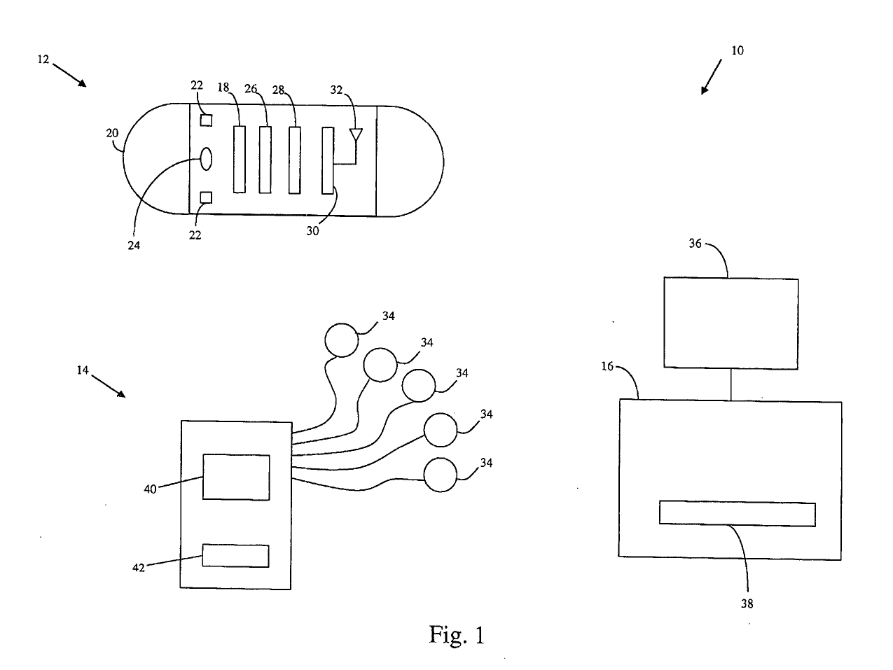

Reference is made to FIG. 1, showing in-vivo sensing system 10 according embodiments of the present invention. The in-vivo sensing system 10 includes an in-vivo sensing device 12, a data recorder 14 and a work station 16. In some embodiments, the in-vivo sensing device 12 may be a wireless device. In some embodiment, the in-vivo sensing device 12 may be autonomous. In some embodiments, the in-vivo sensing device 12 may be a swallowable capsule for sensing the gastrointestinal tract of a patient. However, other body lumens or cavities may be sensed or examined with the in-vivo sensing device 12.

The in-vivo sensing device 12 may include at least one sensor such as an imager 18 for capturing image data in the form of image frames of images of the gastrointestinal tract or other body lumens or cavities, a viewing window 20, one or more illumination sources 22, an optical system 24, a power supply such as a battery 26, a processor 28, a transmitter 30, and an antenna 32 connected to the transmitter 30. As the in-vivo sensing device 12 traverses the gastrointestinal tract or other body lumens, it takes images thereof at a rate of a given number of frames per second. The series of images captured by the imager 18 of the in-vivo sensing device 12 form frames of a video movie. The imager 18 may be and/or contain a CMOS imager. Alternatively, other imagers may be used, e.g. a CCD imager or other imagers.

The image data and or other data captured by the in-vivo sensing device 12 may be transmitted as a data signal by wireless connection, e.g. by wireless communication channel, from the in-vivo sensing device 12 and received by the data recorder 14 via one or more receiving antennas 34, for example an antenna array that may, for example, at least partially surround the patient. The received data signal may be, for example, downloaded to the workstation 16 for processing by a work station processor 38, and for analysis, and display, for example, with a display unit 36. Downloading and/or processing in the workstation 36 may occur off-line for example after the data recorder 14 has completed receiving and recording the data signal received from the in-vivo sensing device 12, or may occur in real-time. In one embodiment of the present invention, the data recorder 14 and the workstation 16 may be integrated into a single unit, for example, may be integrated into a single portable unit. In yet another embodiment of the present invention, the data recorder 14 may include display capability, for example the data recorder 14 may include a viewer 40 for viewing information and/or images, for example information and/or images transmitted by the in-vivo sensing device 12. In another embodiment, processing and/or analysis may be performed at least partially within the data recorder 14 by a data recorder processor 42.

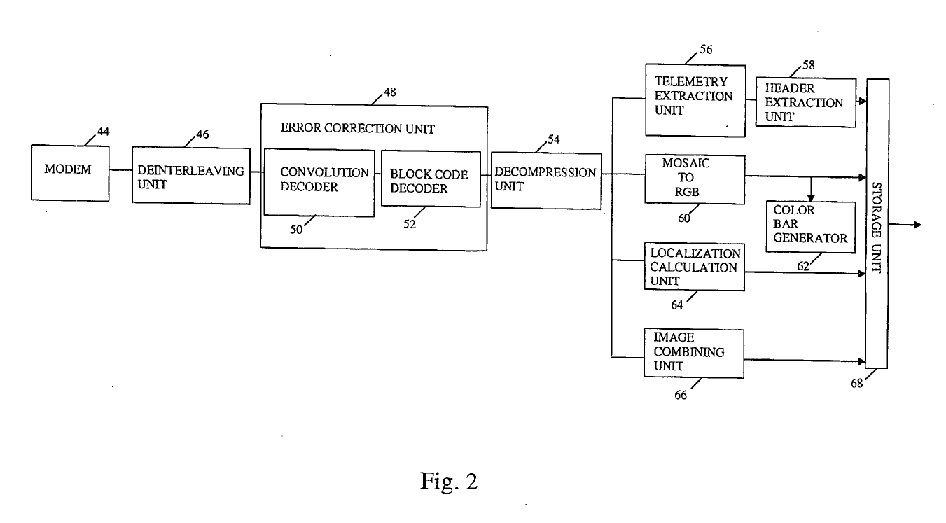

Reference is now made to FIG. 2. The data recorder 14 may include a modem 44 for demodulating the received data signal and a deinterleaving unit 46 configured to deinterleave the demodulated data signal. The data recorder 14 may include an error correction unit 48 for correcting errors, if appropriate, in the deinterleaved signal. The error correction unit 48 may include a convolution decoder 50 for convolution decoding of the deinterleaved data signal. In some embodiments the error correction unit 48 may include a block code decoder 52 for performing block code error detection. In some embodiments the data recorder 14 may comprise a decompression unit 54 for decompressing the error corrected data signal.

The data recorder 14 may include a telemetry extraction unit 56 for extracting telemetry information from the data signal, and a header extraction unit 58, for extracting header information from the telemetry information. Header information may include, for example, the identity of the in-vivo sensing device 12. Apart from header information, telemetry information may include, for example, such information as, the number of frames captured by the imager 18. The data recorder 14 may include a mosaic to RGB interpolation unit 60 to obtain an RGB representation of the image data. In some embodiments, a color bar generator 62 may be included for generating a color bar from the RGB interpolated data. Typically, the color bar consists, for example, of a bar or other shape including a series of colors or a series of areas of color (e.g., a series of stripes or bars of color, arranged to form a large bar or rectangular area), each color corresponding to an image or group of images being displayed, where the color areas are displayed in the same sequence or order that the images or groups of images are typically displayed. Based on the color scheme shown on the color bar a person viewing a series of images taken by the imager of the in-vivo sensing device 12 may identify certain milestones in the anatomy of, for example, the gastrointestinal tract. For example, first gastric image, first duodenal image, cecal valve, first cecal image, etc. A method for creating a color bar is described, for example, in U.S. Patent Application Publication No. 2005/0075551 entitled “System and method for presentation of data streams,” which is incorporated herein by reference.

In accordance with some embodiments, the data recorder 14 includes a localization calculation unit 64 adapted to perform localization calculations for determining the location of the in-vivo sensing device 12 as it moves through the gastrointestinal tract or other body lumens of the patient. A method for determining the location of the in-vivo sensing device 12 is described, for example, in U.S. Patent Application Publication No. 2005/0148816 entitled “Array system and method for locating an in vivo signal source,” which is incorporated herein by reference. The data recorder may include an image combining unit 66 adapted to combine at least some of the image frames of the data signal to form an image of an enlarged field of view. A method for combining image frames to form an image of an enlarged field of view is described, for example, in U.S. Patent Application Publication No. 2003/0045790 entitled “System and method for three dimensional display of body lumens,” which is incorporated herein by reference.

The received data signal may undergo processing by any number or combinations of the various units described above and shown in FIG. 2. The resulting processed data signal may be stored in a storage unit 68 prior to being downloaded to the workstation 16. The storage unit may include one or more memory devices, such as but not limited to random access memory (RAM) devices, read only memory (ROM), programmable read only memory (PROM) devices, electrically programmable read only memory (EPROM) devices, erasable electrically programmable read only memory (EEPROM) devices, flash memory FEPROM devices, or the like, or any suitable combinations of memory devices known in the art.

The present invention also provides methods for recording the data signal received from the in-vivo sensing device 12. According to some embodiments, the method may include, demodulating the data signal, extracting telemetry and header information from the demodulated signal, and storing the processed signal in the storage unit 68. According to some embodiments, the demodulated signal may be deinterleaved. The deinterleaved signal may undergo error correction. Error correction may be performed by convolution decoding. In addition block code correction may be carried out after convolution decoding. In some embodiments, following error correction the data signal may undergo decompressing.

According to some embodiments, the method also includes generating RGB images from mosaic images of the decompressed signal. In addition, a color bar may be generated from the RGB images. The method may also include determining the location of the in-vivo sensing device as it traverses the gastrointestinal tract or other body lumens or cavities in the body of a person who has swallowed the in-vivo sensing device 12. The method may include forming a combined single image from image frames captured by the imager 18 of the in-vivo sensing device 12 to a combined single image in-vivo sensing device 12 of a larger field of view. The method may further include the steps of downloading, typically to a workstation, data that has been initially processed in the data recorder and performing, typically in the workstation, further image processing or other processing on the initially processed data The data may then be displayed, for example, on the workstation screen, as processed images or a movie.

While the present invention has been described with reference to one or more specific embodiments, the description is intended to be illustrative as a whole and is not to be construed as limiting the invention to the embodiments shown. It is appreciated that various modifications may occur to those skilled in the art that, while not specifically shown herein, are nevertheless within the true spirit and scope of the invention.

1. A data recorder for recording a data signal received from an in-vivo sensing device, the data recorder comprising a modem, a telemetry extraction unit, a header extraction unit, and a storage unit, the data signal comprising image data representing images captured by an imager of the in-vivo sensing device.

2. The data recorder according to claim 1, comprising a deinterleaving unit.

3. The data recorder according to claim 1, comprising an error correction unit.

4. The data recorder according to claim 3, wherein the error correction unit comprises a convolution decoder.

5. The data recorder according to claim 3, wherein the error correction unit comprises a block code decoder.

6. The data recorder according to claim 5, comprising a decompression unit.

7. The data recorder according to claim 6, comprising a mosaic to RGB unit.

8. The data recorder according to claim 7, comprising a color bar generator.

9. The data recorder according to claim 6, comprising a localization calculation unit.

10. The data recorder according to claim 6, comprising an image combining unit.

11. A method for recording a data signal received from an in-vivo sensing device comprising:
demodulating the data signal;
extracting telemetry and header information from the demodulated signal; and
storing the demodulated signal and the telemetry and header information.

12. The method for recording a data signal according to claim 11, comprising deinterleaving the demodulated signal.

13. The method for recording a data signal according to claim 12, comprising error correction of the deinterleaved signal.

14. The method for recording a data signal according to claim 13, wherein the error correction comprises convolution decoding.

15. The method for recording a data signal according to claim 13, wherein the error correction comprises block code correction.

16. The method for recording a data signal according to claim 13, comprising decompressing the error corrected signal.

17. The method for recording a data signal according to claim 16, comprising generating RGB images from mosaic images of the decompressed signal.

18. The method for recording a data signal according to claim 17, comprising generating a color bar for the decompressed data signal.

19. The method for recording a data signal according to claim 16, comprising performing a localization calculation on the decompressed data signal.

20. The method for recording a data signal according to claim 16, comprising combining at least some decompressed images frames into a combined single image.
